(19) United States
(12) Patent Application Publication
(10) Pub. No.: US 2022/0304944 A1
(43) Pub. Date: Sep. 29, 2022

(54) METHODS FOR REDUCING THC CONTENT IN COMPLEX CANNABINOID MIXTURES IN WHICH THC IS A MINOR COMPONENT

(71) Applicant: Canopy Growth Corporation, Smiths Falls (CA)

(72) Inventors: Christopher ADAIR, Smiths Falls (CA); Ben GEILING, Smiths Falls (CA); Mohammadmehdi HAGHDOOST MANJILI, Smiths Falls (CA); Anusha Geethangani Perera SAMARANAYAKA, Smiths Falls (CA)

(21) Appl. No.: 17/637,246

(22) PCT Filed: Aug. 21, 2020
(86) PCT No.: PCT/CA2020/051151

§ 371 Date: Feb. 22, 2022

Related U.S. Application Data
(60) Provisional application No. 62/890,982, filed on Aug. 23, 2019.
(60) Provisional application No. 63/015,843, filed on Apr. 27, 2020.

(51) Int. Cl. A61K 31/05

(52) U.S. Cl. CPC A61K 31/05

(57) ABSTRACT

Disclosed herein is a method for upgrading a cannabinoid mixture that comprises tetrahydrocannabinol (THC) and one or more non-THC cannabinoids, when the cannabinoid mixture has a THC content of less than about 20 wt. %. The method comprises contacting the cannabinoid mixture with a benzoquinone reagent under reaction conditions comprising: (i) a reaction temperature that is within a target reaction-temperature range for the benzoquinone reagent and the cannabinoid mixture; and (ii) a reaction time that is within a target reaction-time range for the benzoquinone reagent, the cannabinoid mixture, and the reaction temperature; such that the THC content of the cannabinoid mixture is reduced to a greater extent than that of at least one of the one or more non-THC cannabinoids on a relative wt. % reduction basis.

### CROSS-REFERENCE TO RELATED APPLICATIONS

This application claims priority to and benefit of U.S. Provisional Patent Application Ser. No. 62/890,982 filed on Aug. 23, 2019, and U.S. Provisional Patent Application Ser. No. 63/015,843 filed on Apr. 27, 2020, each of which is hereby incorporated by reference in its entirety.

### TECHNICAL FIELD

The present disclosure generally relates to reducing tetrahydrocannabinol (THC) content in mixtures of cannabinoids. In particular, the present disclosure relates to reducing THC content in mixtures of cannabinoids in which THC is a minor component such as those derived from hemp.

### BACKGROUND

Cannabinoids are a diverse class of compounds that may be characterized in pharmacological terms, chemical-terms, and/or based on their origin. Many cannabinoids are derived from natural sources and, as such, cannabinoids are often provided in complex mixtures that comprise numerous cannabinoids—so called “broad-spectrum” cannabinoid compositions. The number of potential applications of broad-spectrum cannabinoid compositions is increasing rapidly as researchers work to uncover the effects and opportunities that result from such complex mixtures in both medical and recreational contexts.

Tetrahydrocannabinol (THC) is a well-known cannabinoid that is currently being investigated for a wide variety of therapies at least in part due to its psychoactive effects. While the psychoactive effects of THC are central to many medical and recreational applications, there are also numerous applications in which THC—and its associated effects—are not desirable. Such applications typically use source materials that are low in THC (such as those derived from hemp biomass), but the THC content of these materials may still be too high for many purposes. For example, broad-spectrum cannabinoid compositions that comprise less that about 0.3 weight percent THC are desirable, but many hemp extracts have THC contents well above this level. Accordingly, numerous cannabinoid-related applications stand to benefit from methods for reducing the THC content of cannabinoid mixtures having relatively low THC concentrations. In other words, methods for upgrading cannabinoid mixtures are desirable as a means of accessing broad- spectrum cannabinoid compositions with low THC content.

### SUMMARY

Thymoquinone is naturally occurring compound that is currently being investigated due to its potential activity as a hepatoprotective agent, an anti-inflammatory agent, an antioxidant, a cytotoxic agent, and/or an anti-cancer agent. 2,5-dihydroxy-1,4-benzoquinone (DHBQ) is structurally similar to thymoquinone, and it is currently being investigated as a binucleating ligand for assembling coordination polymers. In contrast to the active research in these areas, relatively little work has been done to illustrate how thymoquinone, DHBQ, and related compounds can be utilized in the cannabis space. The present disclosure reports that thymoquinone and DHBQ can be utilized to upgrade cannabinoid mixtures having relatively low THC concentrations by reducing the THC content thereof. More generally, the present disclosure reports that a variety of benzoquinone reagents are useful in this respect, and that such reagents can be utilized to access broad-spectrum cannabinoid compositions having low THC contents by upgrading cannabinoid mixtures with varying degrees of selectivity. Importantly, the experimental results reported herein indicate that benzoquinones can be used to upgrade cannabinoid mixtures having relatively low THC concentrations under relatively mild reaction conditions without requiring harmful solvents such as benzene.

Select embodiments of the present disclosure relate to a method for upgrading a cannabinoid mixture that comprises tetrahydrocannabinol (THC) and one or more non-THC cannabinoids, wherein the cannabinoid mixture has a THC content of less than about 20 wt. %, the method comprising contacting the cannabinoid mixture with a benzoquinone reagent under reaction conditions comprising: (i) a reaction temperature that is within a target reaction-temperature range for the benzoquinone reagent and the cannabinoid mixture; and (ii) a reaction time that is within a target reaction-time range for the benzoquinone reagent, the cannabinoid mixture, and the reaction temperature; such that the THC content of the cannabinoid mixture is reduced to a greater extent than that of at least one of the one or more non-THC cannabinoids on a relative wt. % reduction basis.

Select embodiments of the present disclosure relate to a method for upgrading a cannabinoid mixture that comprises tetrahydrocannabinol (THC) and cannabidiol (CBD), wherein the cannabinoid mixture has a THC content of less than 20 wt.

% and a CBD content of at least about 15 wt. %, the method comprising contacting the cannabinoid mixture with 2,5-dihydroxy-1,4-benzoquinone under reaction conditions comprising: (i) a reaction temperature that is between about 80° C. and about 190° C.; and (ii) a reaction time that is between about 3 h and about 72 h; such that the THC content of the cannabinoid mixture is reduced to a greater extent than the CBD content of the cannabinoid mixture on a relative wt. % reduction basis.

Select embodiments of the present disclosure relate to a method for upgrading a cannabinoid mixture that comprises tetrahydrocannabinol (THC) and cannabidiol (CBD), wherein the cannabinoid mixture has a THC content of less than about 20 wt. % and a CBD content of at least about 15 wt. %, the method comprising contacting the cannabinoid mixture with thymoquinone under reaction conditions comprising: (i) a reaction temperature that is between about 80° C. and about 190° C.; and (ii) a reaction time that is between about 3 h and about 72 h; such that the THC content of the cannabinoid mixture is reduced to a greater extent than the CBD content of the cannabinoid mixture on a relative wt. % reduction basis.

In an embodiment, the present disclosure relates to a method for upgrading a cannabinoid mixture that comprises tetrahydrocannabinol (THC) and cannabidiol (CBD), wherein the cannabinoid mixture has a THC content of less than 20 wt. % and a CBD content of at least about 15 wt. %, the method comprising contacting the cannabinoid mixture with 4-tert-butyl-5-methoxy-1,2-benzoquinone under reaction conditions comprising: (i) a reaction temperature that is between about 70° C. and about 160° C.; and (ii) a reaction time that is between about 3 h and about 48 h; such that the THC content of the cannabinoid mixture is reduced to a greater extent than the CBD content of the cannabinoid mixture on a relative wt. % reduction basis.

In an embodiment, the present disclosure relates to a method for upgrading a cannabinoid mixture that comprises tetrahydrocannabinol (THC) and cannabidiol (CBD), wherein the cannabinoid mixture has a THC content of less than about 20 wt. % and a CBD content of at least about 15 wt. %, the method comprising contacting the cannabinoid mixture with tetrachloro-1,4-benzoquinone under reaction conditions comprising: (i) a reaction temperature that is between about 80° C. and about 180° C.; and (ii) a reaction time that is between about 3 h and about 48 h; such that the THC content of the cannabinoid mixture is reduced to a greater extent than the CBD content of the cannabinoid mixture on a relative wt. % reduction basis.

Other aspects and features of the methods of the present disclosure will become apparent to those ordinarily skilled in the art upon review of the following description of specific embodiments.

### BRIEF DESCRIPTION OF THE DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 shows a process flow charts for executing a method in accordance with the present disclosure.

FIG. 2 shows a process flow charts for executing an alternate method in accordance with the present disclosure.

FIG. 3 shows an HPLC-DAD chromatogram of a hemp-derived Low-THC content input material in accordance with the present disclosure.

FIG. 4 shows an HPLC-DAD chromatogram of an upgraded output material in accordance with the present disclosure.

FIG. 5 shows an HPLC-DAD chromatogram of an unfiltered reaction mixture comprising 2,5-dihydro-1,4-benzoquinone (DHBQ).

FIG. 6 shows a main effects plot for dTHC in a full factorial experiment relating to a method in accordance with the present disclosure.

FIG. 7 shows an interaction effects plot for dTHC in a full factorial experiment relating to a method in accordance with the present disclosure.

FIG. 8 shows a main effects plot for dCBD in a full factorial experiment relating to a method in accordance with the present disclosure.

FIG. 9 shows an interaction effects plot for dCBD in a full factorial experiment relating to a method in accordance with the present disclosure.

### DETAILED DESCRIPTION

As noted above, the present disclosure reports that thymoquinone and 2,5-dihydroxy-1,4-benzoquinone can be utilized to upgrade cannabinoid mixtures having relatively low THC concentrations by reducing the THC content thereof. More generally, the present disclosure reports that a variety of benzoquinone reagents are useful in providing access to broad-spectrum cannabinoid compositions having low THC contents, and that such reagents show varying degrees of selectivity for THC reduction. Without being bound to any particular theory, the present disclosure posits that the ability of benzoquinone reagents to upgrade complex mixtures of cannabinoids as set out herein may be tied to a combination of steric and electronic effects. For example, with respect to steric effects, experiments indicate that naphthoquinones and anthraquinones—which present substantially bulkier steric profiles relative to benzoquinones—are less effective under the conditions investigated, and with respect to electronic effects, experiments suggest that upgrading reactivity may correlate with oxidation potential under the conditions investigated. Importantly, the experimental results reported herein indicate that benzoquinones can be used to upgrade cannabinoid mixtures having relatively low THC concentrations under relatively mild reaction conditions without requiring harmful solvents such as benzene.

Select embodiments of the present disclosure relate to a method for upgrading a cannabinoid mixture that comprises THC and one or more non-THC cannabinoids, wherein the cannabinoid mixture has a THC content of less than about 20 wt. %, the method comprising contacting the cannabinoid mixture with a benzoquinone reagent under reaction conditions comprising: (i) a reaction temperature that is within a target reaction-temperature range for the benzoquinone reagent and the cannabinoid mixture; and (ii) a reaction time that is within a target reaction-time range for the benzoquinone reagent, the cannabinoid mixture, and the reaction temperature; such that the THC content of the cannabinoid mixture is reduced to a greater extent than that of at least one of the one or more non-THC cannabinoids on a relative wt. % reduction basis.

As used herein, the term “upgrade” and its derivatives is intended to refer to reducing the THC content in a cannabinoid mixture that initially comprises at least some

THC. In select embodiments of the present disclosure, the cannabinoid mixture may have a THC content of between: (i) about 0.3 wt. % and about 20.0 wt. %; (ii) about 0.3 wt. % and about 15.0 wt. %; (iii) about 0.3 wt. % and about 10.0 wt. %; or (iv) about 0.3 wt. % and about 5.0 wt. %. In select embodiments of the present disclosure, the THC content of the cannabinoid mixture is reduced to less than 1% w/w, less than 0.3% w/w, or less than 0.1% w/w. A lower THC content may enable the upgraded cannabinoid mixture to avoid regulatory requirements imposed upon products containing THC.

In select embodiments of the present disclosure, the THC content of the cannabinoid mixture is reduced to a greater extent than that of at least one of the one or more non-THC cannabinoids on a relative wt. % reduction basis. In select embodiments of the present disclosure, the one or more non-THC cannabinoids may comprise cannabidiol (CBD), and the THC content of the cannabinoid mixture may be reduced to a greater extent than the CBD content. In select embodiments of the present disclosure, the one or more non-THC cannabinoids may comprise cannabigerol (CBG), and the THC content of the cannabinoid mixture may be reduced to a greater extent than the CBG content. In select embodiments of the present disclosure, the one or more non-THC cannabinoids may comprise cannabichromene (CBC), and the THC content of the cannabinoid mixture may be reduced to a greater extent than the CBD content.

In the context of the present disclosure, a “cannabinoid mixture” is any composition that comprises at least two cannabinoids, and a “broad spectrum cannabinoid composition” is one which contains at least three cannabinoids. In the context of the present disclosure, both cannabinoid mixtures, and broad-spectrum cannabinoid compositions may further comprise non-cannabinoid compounds such as waxes, oils, terpenes, and the like.

As used herein, the term “cannabinoid” refers to: (i) a chemical compound belonging to a class of secondary compounds commonly found in plants of genus cannabis; and/or (ii) one of a class of diverse chemical compounds that may act on cannabinoid receptors such as CB1 and CB2.

In select embodiments of the present disclosure, the cannabinoid is a compound found in a plant, e.g., a plant of genus cannabis, and is sometimes referred to as a phytocannabinoid. One of the most notable cannabinoids of the phytocannabinoids is tetrahydrocannabinol (THC), the primary psychoactive compound in cannabis. Cannabidiol (CBD) is another cannabinoid that is a major constituent of the phytocannabinoids. There are at least 113 different cannabinoids isolated from cannabis, exhibiting varied effects.

In select embodiments of the present disclosure, the cannabinoid is a compound found in a mammal, sometimes called an endocannabinoid.

In many cases, a cannabinoid can be identified because its chemical name will include the text string “*cannabi*”. However, there are a number of cannabinoids that do not use this nomenclature, such as for example those described herein.

As well, any and all isomeric, enantiomeric, or optically active derivatives are also encompassed. In particular, where appropriate, reference to a particular cannabinoid includes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form). As will be appreciated by those skilled in the art who have benefitted from the teachings of the present disclosure, the term “cannabinoid” may refer to: (i) salts of such acid forms, such as Na⁺ or Ca²⁺ salts of such acid forms; and/or (ii) ester forms thereof, such as formed by hydroxyl-group esterification to form traditional esters, sulphonate esters, and/or phosphate esters.

Examples of cannabinoids include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Δ6-Cannabidiol (Δ6-CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinol (THC or Δ9-THC), Δ8-tetrahydrocannabinol (Δ8-THC), trans-Δ10-tetrahydrocannabinol (trans-Δ10-THC), cis-Δ10-tetrahydrocannabinol (cis-Δ10-THC),Tetrahydrocannabinolic acid C4 (THCA-C4), Tetrahydrocannabinol C4 (THC-C4), Tetrahydrocannabivarinic acid (THCVA), Tetrahydrocannabivarin (THCV), Δ8-Tetrahydrocannabivarin (Δ8-THCV), Δ9-Tetrahydrocannabivarin (Δ9-THCV), Tetrahydrocannabiorcolic acid (THCA-C1), Tetrahydrocannabiorcol (THC-C1), Δ7-cis-iso-tetrahydrocannabivarin, Δ8-tetrahydrocannabinolic acid (Δ8-THCA), Δ9-tetrahydrocannabinolic acid (Δ9-THCA), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodivarin (CBDV), Cannabitriol (CBT), 11-hydroxy-Δ9-tetrahydrocannabinol (11-OH-THC), 11-nor 9-carboxy-Δ9-tetrahydrocannabinol, Ethoxy-cannabitriolvarin (CBTVE), 10-Ethoxy-9-hydroxy-Δ6a-tetrahydrocannabinol, Cannabitriolvarin (CBTV), 8,9 Dihydroxy-Δ6a(10a)-tetrahydrocannabinol (8,9-Di-OH-CBT-05), Dehydrocannabifuran (DCBF), Cannbifuran

(CBF), Cannabichromanon (CBCN), Cannabicitran, 10-Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, hexahydrocannibinol, and Dodeca-2E,4E-dienoic acid isobutylamide.

As used herein, the term “THC” refers to tetrahydrocannabinol. “THC” is used interchangeably herein with “Δ9-THC”.

Structural formulae of cannabinoids of the present disclosure may include the following:

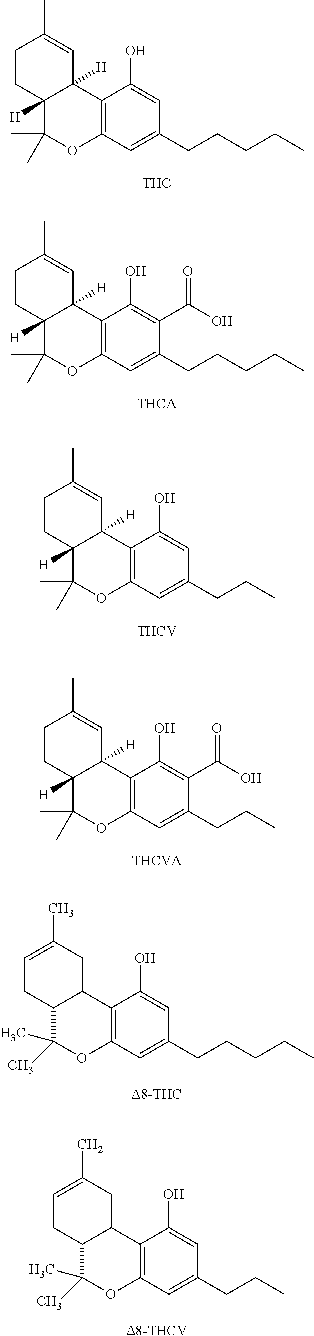

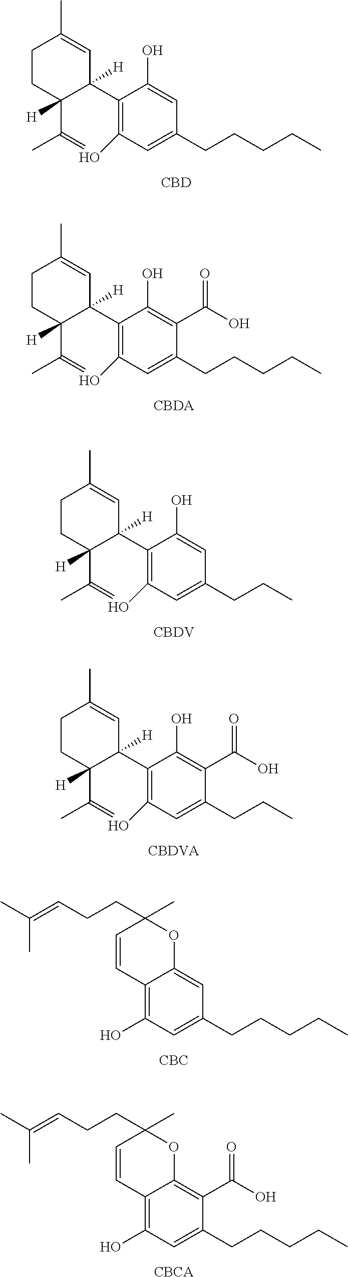

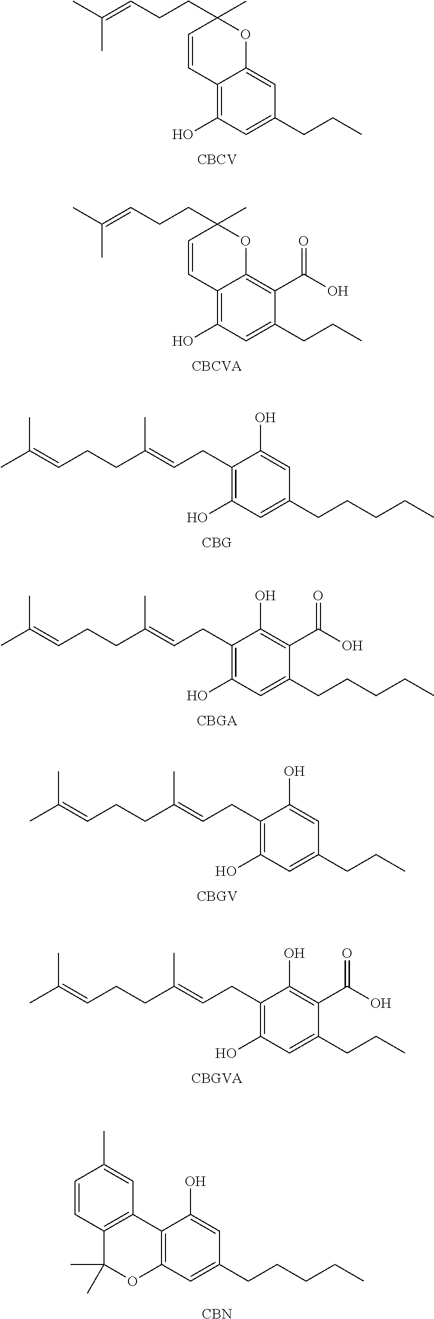

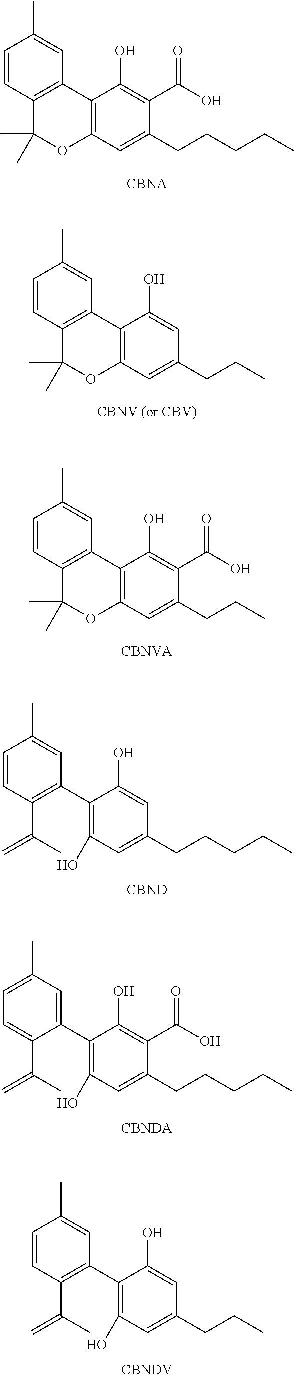

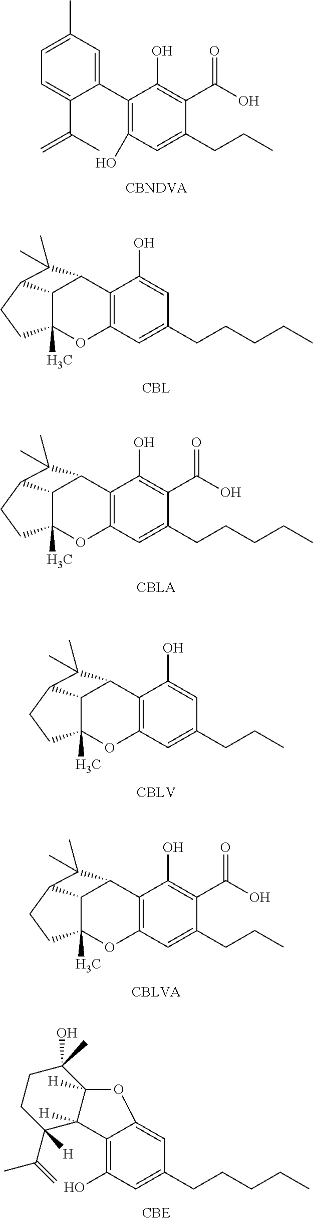

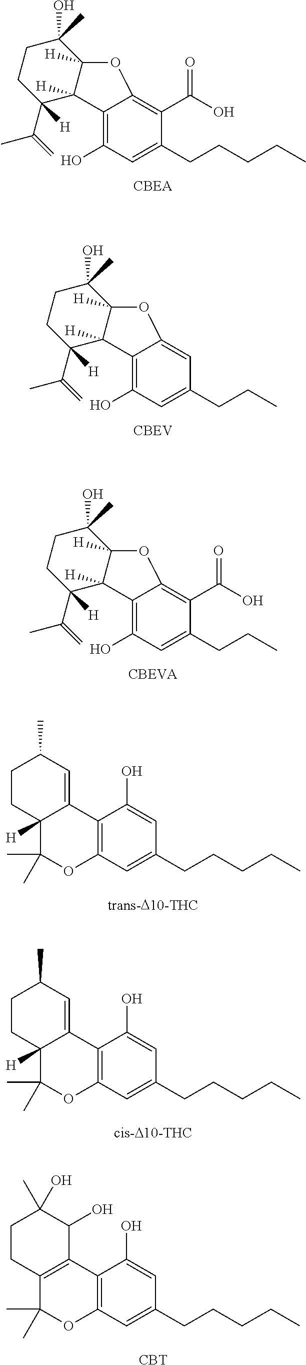

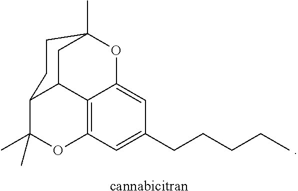

As used herein, the term “non-THC cannabinoids” may refer to any of the cannabinoids described herein that are not THC or any of its homologs or isomers (e.g. Δ8-THC, trans-Δ10-THC, cis-Δ10-THC, THCV, Δ8-THCV, or Δ9-THCV).

In select embodiments, the cannabinoids in the cannabinoid mixture may comprise for example and without limitation be any of those described herein. In a particular, embodiment, the cannabinoid mixture may comprise one or more of THC, Δ8-THC, trans-Δ10-THC, cis-Δ10-THC, THCV, Δ8-THCV, or Δ9-THCV and at least one of CBD, CBDV, CBC, CBCV, CBG, CBGV, CBN, CBNV, CBND, CBNDV, CBE, CBEV, CBL, CBLV, CBT, or cannabicitran.

In select embodiments of the present disclosure, the cannabinoid mixture may comprise THC and/or THCV and at least one of CBD, CBDV, CBC, CBCV, CBG, CBGV, or a regioisomer thereof. As used herein, the term “regioisomers” refers to compounds that differ only in the location of a particular functional group.

In select embodiments of the present disclosure, the cannabinoid mixture may be derived from hemp biomass. In select embodiments of the present disclosure, the cannabinoid mixture may comprise a distillate, a resin, an extract, or a combination thereof.

In select embodiments of the present disclosure, the benzoquinone reagent may comprise a compound as defined in formula (I) or formula (II):

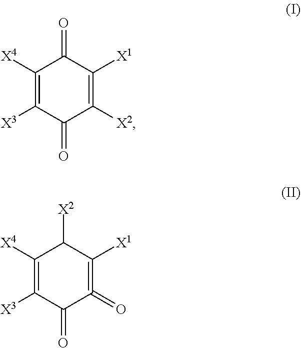

wherein X¹, X², X³, and X⁴ are each independently: H; a halide; a C_{<12}-hydrocarbyl; a C_{<12}-heteroaryl; a C_{<12}-heteroaralkyl; a C_{<12}-heteroaralkenyl; hydroxyl; a C_{<12}-alkoxy; a C_{<12}-amino; a C_{<12}-acyl; a C_{<12}-amide; a C_{<12}-ester; a C_{<12}-ketone; or a substituted analog thereof.

In select embodiments of the present disclosure, the benzoquinone reagent may comprise:

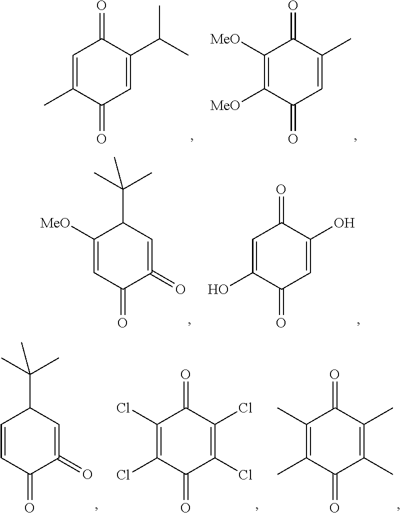

or a combination thereof.

In select embodiments of the present disclosure, the benzoquinone reagent may have an oxidation potential as set out in TABLE 1 which provides oxidation potentials for a series of benzoquinone reagents under non-limiting example conditions. Those skilled in the art who have benefited from the teachings of the present disclosure will readily understand the methods and standards required to determine the oxidation potential of any given benzoquinone reagent. Moreover, those skilled in the art who have benefited from the teaching of the present disclosure will recognize that the oxidation potential of any given benzoquinone reagent may be influenced by external factors such as solvent, pH, solute compositions, solute concentration, and the like.

TABLE 1
Oxidation potentials for a series of benzoquinone reagents under non-limiting example conditions.

| | | | | | E° | E° | E° | E° | E° |
|---|---|---|---|---|---|---|---|---|---|
| X₂ | X₃ | X₅ | X₆ | Σσ | [Q/Q⁻] | [Q⁻/Q²⁻] | [HQ/HQ⁻] | [Q, H⁺/HQ⁻] | [Q, 2H⁺/H₂Q] |
| H | H | H | H | 0.000 | 0.099 | 0.023 | 0.450 | 0.398 | 0.690 |
| C₆H₅ | H | H | H | −0.010 | 0.072 | 0.052 | 0.415 | 0.384 | 0.635 |
| CH₃ | H | H | H | −0.170 | 0.007 | −0.030 | 0.349 | 0.325 | 0.636 |
| C(CH₃)₃ | H | H | H | −0.200 | −0.041 | −0.096 | 0.320 | 0.294 | 0.602 |
| OCH₃ | H | H | H | −0.260 | −0.039 | −0.049 | 0.309 | 0.289 | 0.571 |
| N(CH₃)₂ | H | H | H | −0.830 | −0.221 | −0.144 | 0.124 | 0.182 | 0.466 |
| NH₂ | H | H | H | −0.660 | −0.193 | −0.117 | 0.042 | 0.175 | 0.456 |
| CH₂CH₃ | H | H | H | −0.150 | −0.025 | −0.068 | 0.321 | 0.300 | 0.605 |
| OH | H | H | H | −0.370 | 0.013 | −0.025 | 0.333 | 0.320 | 0.605 |
| OCH₂CH₃ | H | H | H | −0.280 | −0.070 | −0.069 | 0.300 | 0.271 | 0.541 |
| F | H | H | H | 0.340 | 0.231 | 0.153 | 0.559 | 0.467 | 0.687 |
| Cl | H | H | H | 0.370 | 0.242 | 0.195 | 0.595 | 0.491 | 0.706 |
| Br | H | H | H | 0.390 | 0.243 | 0.191 | 0.618 | 0.507 | 0.672 |
| SH | H | H | H | 0.150 | 0.110 | 0.086 | 0.436 | 0.403 | 0.665 |
| SiH₃ | H | H | H | 0.100 | 0.156 | 0.070 | 0.493 | 0.423 | 0.657 |
| CHO | H | H | H | 1.030 | 0.393 | 0.362 | 0.635 | 0.650 | 0.905 |
| COOCH₃ | H | H | H | 0.750 | 0.339 | 0.260 | 0.594 | 0.635 | 0.866 |
| CF₃ | H | H | H | 0.540 | 0.365 | 0.263 | 0.716 | 0.584 | 0.733 |
| CN | H | H | H | 1.000 | 0.479 | 0.401 | 0.853 | 0.686 | 0.778 |
| COOH | H | H | H | 0.770 | 0.592 | −0.068 | 0.621 | 0.644 | 0.799 |
| SO3- | H | H | H | 0.580 | 0.184 | 0.160 | 0.504 | 0.502 | 0.776 |
| NO2 | H | H | H | 1.270 | 0.613 | 0.688 | 1.007 | 0.833 | 0.938 |
| COCH₃ | H | H | H | 0.840 | 0.276 | 0.299 | 0.573 | 0.640 | 0.879 |
| C₆H₅ | C₆H₅ | H | H | −0.020 | 0.012 | 0.008 | 0.381 | 0.339 | 0.607 |
| CH₃ | CH₃ | H | H | −0.340 | −0.090 | −0.133 | 0.297 | 0.262 | 0.564 |
| C(CH₃)₃ | C(CH₃)₃ | H | H | −0.400 | −0.385 | −0.249 | 0.099 | 0.047 | 0.355 |
| OCH₃ | OCH₃ | H | H | −0.520 | −0.048 | 0.065 | 0.404 | 0.333 | 0.563 |
| N(CH₃)₂ | N(CH₃)₂ | H | H | −1.660 | −0.301 | −0.117 | 0.236 | 0.119 | 0.398 |
| NH₂ | NH₂ | H | H | −1.320 | −0.172 | −0.144 | 0.101 | 0.152 | 0.384 |
| CH₂CH₃ | CH₂CH₃ | H | H | −0.300 | −0.113 | −0.118 | 0.257 | 0.238 | 0.549 |
| OH | OH | H | H | −0.740 | 0.041 | 0.028 | 0.370 | 0.339 | 0.527 |
| OCH₂CH₃ | OCH₂CH₃ | H | H | −0.560 | −0.086 | 0.137 | 0.373 | 0.340 | 0.581 |
| F | F | H | H | 0.680 | 0.374 | 0.282 | 0.706 | 0.526 | 0.671 |
| Cl | Cl | H | H | 0.740 | 0.342 | 0.320 | 0.726 | 0.524 | 0.663 |
| Br | Br | H | H | 0.780 | 0.330 | 0.315 | 0.699 | 0.536 | 0.681 |
| SH | SH | H | H | 0.300 | 0.112 | 0.851 | 0.271 | 0.349 | 0.571 |
| SiH₃ | SiH₃ | H | H | 0.200 | 0.191 | 0.237 | 0.589 | 0.450 | 0.645 |
| CHO | CHO | H | H | 2.060 | 0.658 | 0.835 | 1.064 | 0.942 | 0.974 |
| COOCH₃ | COOCH₃ | H | H | 1.500 | 0.445 | 0.417 | 0.732 | 0.707 | 0.866 |
| CF₃ | CF₃ | H | H | 0.540 | 0.365 | 0.263 | 0.716 | 0.584 | 0.733 |
| CN | CN | H | H | 2.000 | 0.886 | 0.856 | 1.210 | 0.914 | 0.912 |
| COOH | COOH | H | H | 1.540 | 0.770 | 0.125 | 0.819 | 0.766 | 0.817 |
| SO3- | SO3- | H | H | 1.160 | 0.184 | 0.265 | 0.535 | 0.600 | 0.798 |
| NO2 | NO2 | H | H | 2.540 | 0.983 | 1.378 | 1.460 | 1.115 | 1.007 |
| COCH₃ | COCH₃ | H | H | 1.680 | 0.421 | 0.433 | 0.833 | 0.689 | 0.788 |
| C₆H₅ | H | C₆H₅ | H | −0.020 | 0.041 | 0.104 | 0.404 | 0.351 | 0.634 |
| CH₃ | H | CH₃ | H | −0.340 | −0.092 | −0.081 | 0.348 | 0.285 | 0.574 |
| C(CH₃)₃ | H | C(CH₃)₃ | H | −0.400 | −0.193 | −0.193 | 0.201 | 0.185 | 0.520 |
| OCH₃ | H | OCH₃ | H | −0.520 | −0.146 | −0.233 | 0.120 | 0.133 | 0.459 |
| N(CH₃)₂ | H | N(CH₃)₂ | H | −1.660 | −0.602 | −0.284 | −0.043 | −0.072 | 0.288 |
| NH₂ | H | NH₂ | H | −1.320 | −0.614 | −0.360 | −0.233 | −0.178 | 0.116 |
| CH₂CH₃ | H | CH₂CH₃ | H | −0.300 | −0.172 | −0.168 | 0.214 | 0.188 | 0.514 |
| OH | H | OH | H | −0.740 | −0.142 | −0.108 | 0.237 | 0.196 | 0.485 |
| OCH₂CH₃ | H | OCH₂CH₃ | H | −0.560 | −0.285 | −0.190 | 0.099 | 0.090 | 0.385 |
| F | H | F | H | 0.680 | 0.344 | 0.270 | 0.691 | 0.509 | 0.667 |
| Cl | H | Cl | H | 0.740 | 0.372 | 0.356 | 0.751 | 0.547 | 0.718 |
| Br | H | Br | H | 0.780 | 0.377 | 0.352 | 0.744 | 0.569 | 0.730 |
| SH | H | SH | H | 0.300 | 0.100 | 0.136 | 0.486 | 0.368 | 0.615 |
| SiH₃ | H | SiH₃ | H | 0.200 | 0.194 | 0.151 | 0.545 | 0.445 | 0.675 |
| CHO | H | CHO | H | 2.060 | 0.628 | 0.569 | 0.953 | 0.858 | 1.083 |
| COOCH₃ | H | COOCH₃ | H | 1.500 | 0.490 | 0.398 | 0.841 | 0.786 | 1.058 |
| CF₃ | H | CF₃ | H | 1.080 | 0.614 | 0.487 | 0.959 | 0.712 | 0.803 |
| CN | H | CN | H | 2.000 | 0.814 | 0.720 | 1.149 | 0.852 | 0.876 |
| COOH | H | COOH | H | 1.540 | 0.997 | −0.252 | 0.901 | 0.812 | 0.924 |
| SO3- | H | SO3- | H | 1.160 | 0.307 | 0.270 | 0.637 | 0.599 | 0.889 |
| NO2 | H | NO2 | H | 2.540 | 0.981 | 0.975 | 1.362 | 1.081 | 1.128 |
| COCH₃ | H | COCH₃ | H | 1.680 | 0.463 | 0.363 | 0.718 | 0.739 | 1.076 |
| C₆H₅ | H | H | C₆H₅ | −0.020 | 0.019 | 0.070 | 0.364 | 0.345 | 0.599 |
| CH₃ | H | H | CH₃ | −0.340 | −0.088 | −0.095 | 0.241 | 0.258 | 0.553 |
| C(CH₃)₃ | H | H | C(CH₃)₃ | −0.400 | −0.192 | −0.274 | 0.124 | 0.157 | 0.467 |
| OCH₃ | H | H | OCH₃ | −0.520 | −0.154 | −0.123 | 0.148 | 0.215 | 0.493 |
| N(CH₃)₂ | H | H | N(CH₃)₂ | −1.660 | −0.468 | −0.255 | −0.017 | 0.037 | 0.338 |
| NH₂ | H | H | NH₂ | −1.320 | −0.345 | −0.265 | −0.143 | 0.020 | 0.285 |
| CH₂CH₃ | H | H | CH₂CH₃ | −0.300 | −0.142 | −0.143 | 0.199 | 0.204 | 0.506 |
| OH | H | H | OH | −0.740 | −0.034 | −0.060 | 0.263 | 0.269 | 0.518 |
| OCH₂CH₃ | H | H | OCH₂CH₃ | −0.560 | −0.173 | −0.167 | 0.164 | 0.175 | 0.438 |
| F | H | H | F | 0.680 | 0.382 | 0.286 | 0.679 | 0.551 | 0.675 |
| Cl | H | H | Cl | 0.740 | 0.389 | 0.350 | 0.745 | 0.584 | 0.683 |
| Br | H | H | Br | 0.780 | 0.387 | 0.358 | 0.776 | 0.616 | 0.734 |
| SH | H | H | SH | 0.300 | 0.135 | 0.149 | 0.439 | 0.402 | 0.548 |
| SiH₃ | H | H | SiH₃ | 0.200 | 0.203 | 0.148 | 0.569 | 0.474 | 0.615 |
| CHO | H | H | CHO | 2.060 | 0.634 | 0.673 | 0.990 | 0.880 | 1.021 |
| COOCH₃ | H | H | COOCH₃ | 1.500 | 0.518 | 0.437 | 0.775 | 0.740 | 0.939 |
| CF3 | H | H | CF3 | 1.080 | 0.620 | 0.496 | 1.025 | 0.785 | 0.797 |
| CN | H | H | CN | 2.000 | 0.815 | 0.734 | 1.285 | 0.970 | 0.874 |
| COOH | H | H | COOH | 1.540 | 0.988 | −0.106 | 0.809 | 0.788 | 0.847 |
| SO3- | H | H | SO3- | 1.160 | 0.302 | 0.269 | 0.614 | 0.574 | 0.810 |
| NO2 | H | H | NO2 | 2.540 | 0.944 | 1.081 | 1.488 | 1.102 | 1.047 |
| COCH₃ | H | H | COCH₃ | 1.680 | 0.375 | 0.513 | 0.740 | 0.720 | 0.926 |
| C₆H₅ | C₆H₅ | C₆H₅ | H | −0.030 | −0.024 | 0.014 | 0.334 | 0.324 | 0.588 |
| CH₃ | CH₃ | CH₃ | H | −0.510 | −0.211 | −0.192 | 0.162 | 0.158 | 0.485 |
| C(CH₃)₃ | C(CH₃)₃ | C(CH₃)₃ | H | −0.600 | −0.560 | −0.468 | −0.088 | −0.079 | 0.229 |
| OCH₃ | OCH₃ | OCH₃ | H | −0.780 | −0.213 | −0.010 | 0.233 | 0.213 | 0.455 |
| N(CH₃)₂ | N(CH₃)₂ | N(CH₃)₂ | H | −2.490 | −0.699 | −0.262 | −0.136 | −0.027 | 0.370 |
| NH₂ | NH₂ | NH₂ | H | −1.980 | −0.556 | −0.361 | −0.163 | −0.129 | 0.120 |
| CH₂CH₃ | CH₂CH₃ | CH₂CH₃ | H | −0.450 | −0.223 | −0.205 | 0.125 | 0.154 | 0.491 |
| OH | OH | OH | H | −1.110 | −0.079 | −0.030 | 0.246 | 0.235 | 0.444 |
| OCH₂CH₃ | OCH₂CH₃ | OCH₂CH₃ | H | −0.840 | −0.290 | 0.048 | 0.236 | 0.205 | 0.465 |
| F | F | F | H | 1.110 | 0.499 | 0.405 | 0.824 | 0.606 | 0.691 |
| Cl | Cl | Cl | H | 1.170 | 0.472 | 0.472 | 0.877 | 0.626 | 0.698 |
| Br | Br | Br | H | 0.450 | 0.462 | 0.477 | 0.848 | 0.643 | 0.720 |
| SH | SH | SH | H | 0.450 | 0.117 | 0.217 | 0.511 | 0.407 | 0.491 |
| SiH₃ | SiH₃ | SiH₃ | H | 0.300 | 0.233 | 0.272 | 0.611 | 0.475 | 0.611 |
| CHO | CHO | CHO | H | 3.090 | 0.796 | 0.978 | 1.257 | 1.072 | 1.167 |
| COOCH₃ | COOCH₃ | COOCH₃ | H | 2.250 | 0.586 | 0.559 | 0.938 | 0.849 | 1.053 |
| CF₃ | CF₃ | CF₃ | H | 1.620 | 0.845 | 0.748 | 1.292 | 0.918 | 0.875 |
| CN | CN | CN | H | 3.000 | 1.178 | 1.122 | 1.553 | 1.134 | 0.968 |
| COOH | COOH | COOH | H | 2.310 | 1.149 | −0.065 | 1.060 | 0.929 | 0.966 |
| SO3- | SO3- | SO3- | H | 1.740 | 0.256 | 0.353 | 0.646 | 0.665 | 0.902 |
| NO2 | NO2 | NO2 | H | 3.810 | 1.261 | 1.510 | 1.701 | 1.269 | 1.147 |
| COCH₃ | COCH₃ | COCH₃ | H | 2.520 | 0.557 | 0.518 | 0.935 | 0.865 | 0.898 |
| C₆H₅ | C₆H₅ | C₆H₅ | C₆H₅ | −0.040 | −0.084 | 0.009 | 0.367 | 0.281 | 0.561 |
| CH₃ | CH₃ | CH₃ | CH₃ | −0.040 | −0.084 | 0.009 | 0.367 | 0.281 | 0.561 |
| C(CH₃)₃ | C(CH₃)₃ | C(CH₃)₃ | C(CH₃)₃ | −0.800 | −1.107 | −0.804 | −0.388 | −0.509 | −0.153 |
| OCH₃ | OCH₃ | OCH₃ | OCH₃ | −1.040 | −0.229 | 0.111 | 0.370 | 0.220 | 0.465 |
| N(CH₃)₂ | N(CH₃)₂ | N(CH₃)₂ | N(CH₃)₂ | −3.320 | −0.629 | −0.322 | −0.253 | −0.138 | 0.203 |
| NH₂ | NH₂ | NH₂ | NH₂ | −2.640 | −0.571 | −0.456 | −0.197 | −0.192 | 0.028 |
| CH₂CH₃ | CH₂CH₃ | CH₂CH₃ | CH₂CH₃ | −0.600 | −0.372 | −0.347 | 0.066 | 0.032 | 0.384 |
| OH | OH | OH | OH | −1.480 | −0.077 | −0.039 | 0.295 | 0.183 | 0.379 |
| OCH₂CH₃ | OCH₂CH₃ | OCH₂CH₃ | OCH₂CH₃ | −1.120 | −0.305 | 0.238 | 0.388 | 0.290 | 0.527 |
| F | F | F | F | 1.360 | 0.638 | 0.531 | 0.986 | 0.670 | 0.731 |
| Cl | Cl | Cl | Cl | 1.480 | 0.564 | 0.588 | 1.003 | 0.663 | 0.684 |
| Br | Br | Br | Br | 1.560 | 0.539 | 0.581 | 0.960 | 0.660 | 0.720 |
| SH | SH | SH | SH | 0.600 | 0.111 | 0.279 | 0.526 | 0.342 | 0.453 |
| SiH₃ | SiH₃ | SiH₃ | SiH₃ | 0.400 | 0.247 | 0.322 | 0.675 | 0.459 | 0.558 |
| CHO | CHO | CHO | CHO | 4.120 | 0.873 | 1.005 | 1.319 | 1.099 | 1.221 |
| COOCH₃ | COOCH₃ | COOCH₃ | COOCH₃ | 3.000 | 0.744 | 0.680 | 1.064 | 0.909 | 1.052 |
| CF₃ | CF₃ | CF₃ | CF₃ | 2.160 | 0.972 | 0.902 | 1.397 | 0.937 | 0.833 |
| CN | CN | CN | CN | 4.000 | 1.48 | 1.430 | 1.832 | 1.271 | 1.025 |
| COOH | COOH | COOH | COOH | 3.080 | 1.278 | 0.068 | 1.143 | 0.970 | 0.980 |
| SO3- | SO3- | SO3- | SO3- | 2.320 | 0.084 | 0.348 | 0.613 | 0.546 | 0.846 |
| NO₂ | NO₂ | NO₂ | NO₂ | 5.080 | 1.613 | 1.662 | 1.939 | 1.441 | 1.231 |
| COCH₃ | COCH₃ | COCH₃ | COCH₃ | 3.360 | 0.663 | 0.657 | 0.914 | 0.768 | 0.865 |
| CN | CN | Cl | Cl | 2.740 | 1.096 | 1.079 | 1.461 | 1.027 | 0.884 |

In the context of the present disclosure, the term “contacting” and its derivatives is intended to refer to brining the cannabinoid mixture and the benzoquinone reagent as disclosed herein into proximity such that a chemical reaction can occur. In some embodiments, the contacting may be by adding the benzoquinone reagent to the cannabinoid mixture. In some embodiments, the contacting may be by combining, mixing, or both.

In select embodiments of the present disclosure, the contacting of the cannabinoid mixture with the benzoquinone reagent comprises introducing the benzoquinone reagent to the cannabinoid mixture at a benzoquinone:THC ratio of between: (i) about 1.0:1.0 and about 20.0:1.0 on a molar basis; (ii) about 1.0:1.0 and about 15.0:1.0 on a molar basis; (iii) about 1.0:1.0 and about 1.0:10.0 on a molar basis; or (iv) about 3.0:1.0 and about 7.0:1.0 on a molar basis. In a particular embodiment, the benzoquinone:THC ratio is about 5.5:1.0, about 5.6:1.0, about 5.7:1.0, about 5.8:1.0, about 5.9:1.0, about 6.0:1.0, about 6.1:1.0, about 6.2:1.0, about 6.3:1.0, about 6.4:1.0, about 6.5:1.0, about 6.6:1.0, about 6.7:1.0, about 6.8:1.0, about 6.9:1.0 or about 7.0:1.0. In another particular embodiment, the benzoquinone:THC ratio is about 12.5:1.0, about 12.6:1.0, about 12.7:1.0, about 12.8:1.0, about 12.9:1.0, about 13.0:1.0, about 13.1:1.0, about 13.2:1.0, about 13.3:1.0, about 13.4:1.0, or about 13.5:1.0.

In select embodiments of the present disclosure, the benzoquinone reagent (both spent and unreacted) may be separated from the crude product mixture and reactivated such that it may be reused in a further reaction. Those skilled in the art who have benefitted from the teachings of the present disclosure will recognize suitable methods for regenerating the benzoquinone reagent such as treatment with a strong reductant.

In the context of the present disclosure, the relative quantities of cannabinoids may be expressed as a ratio such as THC:non-THC cannabinoid. Those skilled in the art will recognize that a variety of analytical methods may be used to determine such ratios, and the protocols required to implement any such method are within the purview of those skilled in the art. By way of non-limiting example, such ratios may be determined by diode-array-detector high pressure liquid chromatography, UV-detector high pressure liquid chromatography, nuclear magnetic resonance spectroscopy, mass spectroscopy, flame-ionization gas chromatography, gas chromatograph-mass spectroscopy, or combinations thereof.

In select embodiments of the present disclosure, the target reaction-temperature range may be between: (i) about 25° C. and about 200° C.; (ii) about 50° C. and about 160° C.; (iii) about 80° C. and about 140° C.; or (iv) about 90° C. and about 130° C. In a particular embodiment, the target reaction-temperature is about 110° C., 111° C., 112° C., 113° C., 114° C., 115° C., 116° C., 117° C., 118° C., 119° C., 120° C., 121° C., 122° C., 123° C., 124° C., 125° C., 126° C., 127° C., 128° C., 129° C., or 130° C. Those skilled in the art who have benefitted from the teachings of the present disclosure will recognize that selecting a target-reaction temperature range may be done having regard to the particulars of the input material, the desired extent of upgrading, the particulars of the benzoquinone reagent, the particulars of the solvent system (or lack thereof), the reaction time, and the like. In particular, those skilled in the art who have benefitted from the teachings of the present disclosure may adapt the full factorial experimental protocol set out in the examples of Series A to select suitable experimental parameters.

In select embodiments of the present disclosure, the target reaction-time range is between: (i) about 0.5 h and about 72 h; (ii) about 5 h and about 60 h; (iii) about 22 h and about 48 h; or (iv) about 24 h and about 30 h. In a particular embodiment, the target reaction-time is about 2 h, about 3 h, about 4 h, about 5 h, about 6 h, about 7 h, about 8 h, about 9 h, about 10 h, about 11 h, about 12 h, about 13 h, about 14 h, about 15 h, about 16 h, about 17 h, about 18 h, about 19 h, about 20 h, about 21 h, about 22 h, about 23 h, or about 24 h. Those skilled in the art who have benefitted from the teachings of the present disclosure will recognize that selecting a target-reaction time range may be done having regard to the particulars of the input material, the desired extent of upgrading, the particulars of the benzoquinone reagent, the particulars of the solvent system (or lack thereof), the reaction temperature, and the like. In particular, those skilled in the art who have benefitted from the teachings of the present disclosure may adapt the full factorial experimental protocol set out in the examples of Series A to select suitable experimental parameters.

In select embodiments of the present disclosure, the contacting of the cannabinoid mixture with the benzoquinone reagent may be executed neat or in the presence of a solvent. The examples set out below in Series A indicate that neat reaction conditions may be suitable under the conditions evaluated. Experiments related to those set out in the examples set out below in Series A indicate that solvents may be suitable under similar reaction conditions—particularly when the solvent is aprotic and when the reaction is executed at elevated pressure reaction. More generally, in instances where a solvent is employed, the solvent may be protic or aprotic. By way of non-limiting example, an aprotic- solvent system may comprise dimethyl sulfoxide, ethyl acetate, dichloromethane, chloroform, toluene, pentane, heptane, hexane, diethyl ether, tert-butyl methyl ether, tetrahydrofuran, dioxane, dimethylformamide, dimethylacetamide, N-methylpyrrolidone, anisole, butyl acetate, cumene, ethyl formate, isobutyl acetate, isopropyl acetate, methyl acetate, methylethylketone, methylisobutylketone, propyl acetate, cyclohexane, para-xylene, meta-xylene, ortho-xylene, 1,2-dichloroethane, or a combination thereof. As will be appreciated by those skilled in the art who have benefitted from the present disclosure, aprotic solvent systems may comprise small amounts of protic species, the quantities of which may be influenced by the extent to which drying and/or degassing procedures are employed. By way of non-limiting example a protic-solvent system may comprise methanol, ethanol, 1-propanol, 2-propanol, 1-butanol, 2-butanol, water, acetic acid, formic acid, 3-methyl-1-butanol, 2-methyl-1-propanol, 1-pentanol, nitromethane, or a combination thereof.

In select embodiments of the present disclosure, the contacting of the cannabinoid mixture with the benzoquinone reagent is in the presence of oxygen. The atmospheric oxygen may be bubbled through the cannabinoid mixture and/or the cannabinoid mixture may be exposed to the air.

Select embodiments of the present disclosure relate to a method for upgrading a cannabinoid mixture that comprises THC and CBD, wherein the cannabinoid mixture has a THC content of less than 20 wt. % and a CBD content of at least about 15 wt. %, the method comprising contacting the cannabinoid mixture with 2,5-dihydro-1,4-benzoquinone (DHBQ) under reaction conditions comprising: (i) a reaction temperature that is between about 90° C. and about 180° C.; and (ii) a reaction time that is between about 3 h and about 48 h; such that the THC content of the cannabinoid mixture is reduced to a greater extent than the CBD content of the cannabinoid mixture on a relative wt. % reduction basis.

Select embodiments of the present disclosure relate to a method for upgrading a cannabinoid mixture that comprises THC and CBD, wherein the cannabinoid mixture has a THC content of less than about 20 wt. % and a CBD content of at least about 15 wt. %, the method comprising contacting the cannabinoid mixture with thymoquinone under reaction conditions comprising: (i) a reaction temperature that is between about 80° C. and about 190° C.; and (ii) a reaction time that is between about 3 h and about 72 h; such that the THC content of the cannabinoid mixture is reduced to a greater extent than the CBD content of the cannabinoid mixture on a relative wt. % reduction basis.

Select embodiments of the present disclosure relate to a method for upgrading a cannabinoid mixture that comprises THC and CBD, wherein the cannabinoid mixture has a THC content of less than 20 wt. % and a CBD content of at least about 15 wt. %, the method comprising contacting the cannabinoid mixture with 4-tert-butyl-5-methoxy- 1,2-benzoquinone under reaction conditions comprising: (i) a reaction temperature that is between about 70° C. and about 160° C.; and (ii) a reaction time that is between about 3 h and about 48 h; such that the THC content of the cannabinoid mixture is reduced to a greater extent than the CBD content of the cannabinoid mixture on a relative wt. % reduction basis.

Select embodiments of the present disclosure relate to a method for upgrading a cannabinoid mixture that comprises THC and CBD, wherein the cannabinoid mixture has a THC content of less than about 20 wt. % and a CBD content of at least about 15 wt. %, the method comprising contacting the cannabinoid mixture with tetrachloro-1,4- benzoquinone under reaction conditions comprising: (i) a reaction temperature that is between about 80° C. and about 180° C.; and (ii) a reaction time that is between about 3 h and about 48 h; such that the THC content of the cannabinoid mixture is reduced to a greater extent than the CBD content of the cannabinoid mixture on a relative wt. % reduction basis.

In the context of the present disclosure, reducing the THC content to a greater extent than that of at least non-THC cannabinoid one cannabinoid may yield a product mixture in which the THC content has been reduced by: (i) at least about 10% on a molar basis relative to the input material; (ii) at least about 25% on a molar basis relative to the input material; (iii) at least about 45% on a molar basis relative to the input material; or (iv) at least about 70% on a molar basis relative to the input material. In each case, the content of one or more non-THC cannabinoids may also be reduced, but the content of at least one non-THC cannabinoid will be reduced to a lesser extent than THC. For example, a method in accordance with the present disclosure may reduce the THC content of a cannabinoid mixture by 50% on a molar basis relative to the input material.

In the context of the present disclosure, reducing the THC content of cannabinoid mixture may equate to oxidizing THC in the mixture to cannabinol (CBN). Accordingly, increases in the CBN content of a mixture of cannabinoids may result from the methods of the present disclosure.

### EXAMPLES

### Series A

The following examples describe a series of experiments in which complex cannabinoid mixtures having low THC contents were contacted with DHBQ to reduce the THC content of the complex cannabinoid mixtures as generally characterized in SCHEME 1.

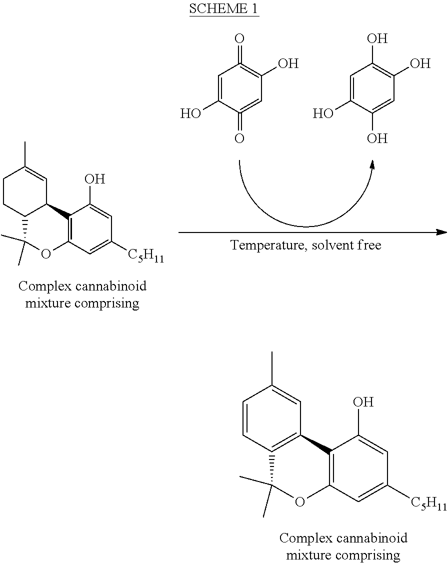

An archetypal experimental protocol for implementing the transformation of SCHEME 1 in accordance with a method of the present disclosure is as follows.

In a first step, a reaction vessel is charged with a hemp-derived input material (such as a primary solvent extract or a distillate) and heated to about 80° C. (to reduce its viscosity, for example).

In a second step, DHBQ powder is added to the heated cannabinoid mixture in a quantity sufficient to provide a DHBQ ratio of about 6:1 on a molar basis.

In a third step, the reaction vessel is heated to about 125° C. in the absence of exogenous solvent for about 18 hours with gentle stirring (e.g. 125 rpm). During this step, a small quantity of the reaction mixture may be withdrawn and analyzed in order to monitor the reaction process.

In a fourth step, the reaction mixture is filtered hot to obtain crude output material which may be analyzed to determine the quantity of cannabinoids and/or to confirm the presence/absence of DHBQ and/or a reduced form thereof.

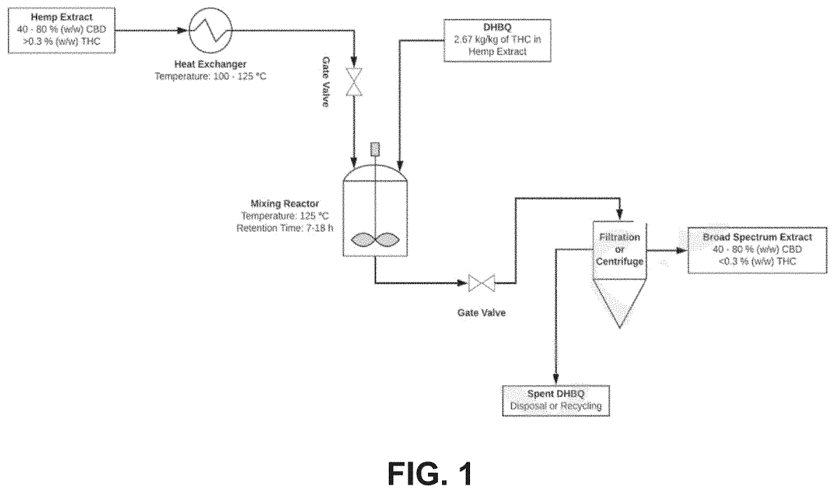

A process flow charts for the foregoing experimental protocol is set out in FIG. 1.

In an alternate archetypal experimental protocol, the first, second and third steps are executed as set above, but the fourth step is different. In the alternate fourth step, the reaction mixture is cooled to room temperature and diluted with about 0.5 L of heptane per kg of hemp input material. After stirring for about 30 minutes at room temperature, the solution is filtered to obtain diluted crude output material which is heated to between about 60° C. and about 70° C. under reduced pressure (such as in a wipe-film evaporator or other continuous evaporation system) to substantially remove the heptane, and then the remaining residue is analyzed to determine the quantity of cannabinoids and/or to confirm the presence/absence of DHBQ and/or a reduced form thereof.

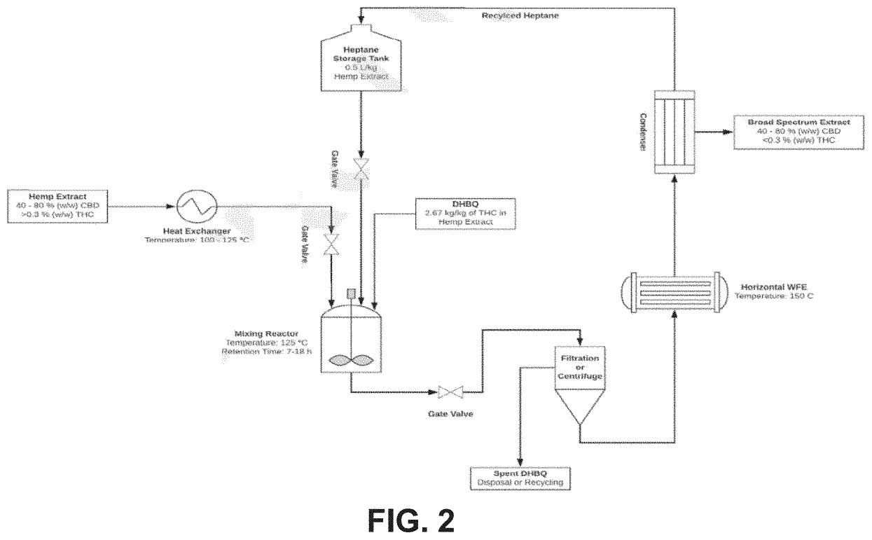

A process flow chart for the foregoing alternative experimental protocol is set out in FIG. 2.

In a representative experiment based on the process flow chart in FIG. 1, a hemp-derived input material was upgraded in accordance with a method of the present disclosure, and the amount of THC in the material was reduced from 2.61% (w/w) to a value below the instrument quantification limit (<LoQ) of the HPLC. At the same time, the amount of CBD in the material was reduced from 75.57% (w/w) to 71.16% (w/w) indicating that approximately 94% of CBD remained intact during the process.

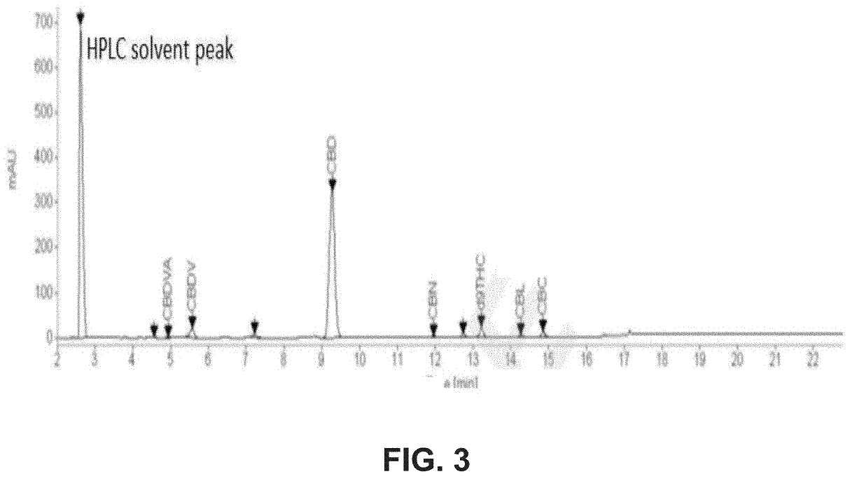

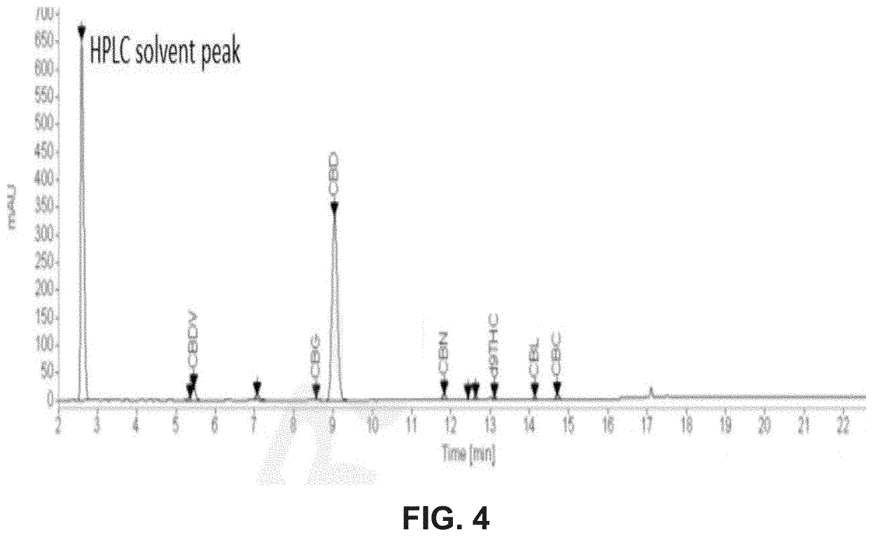

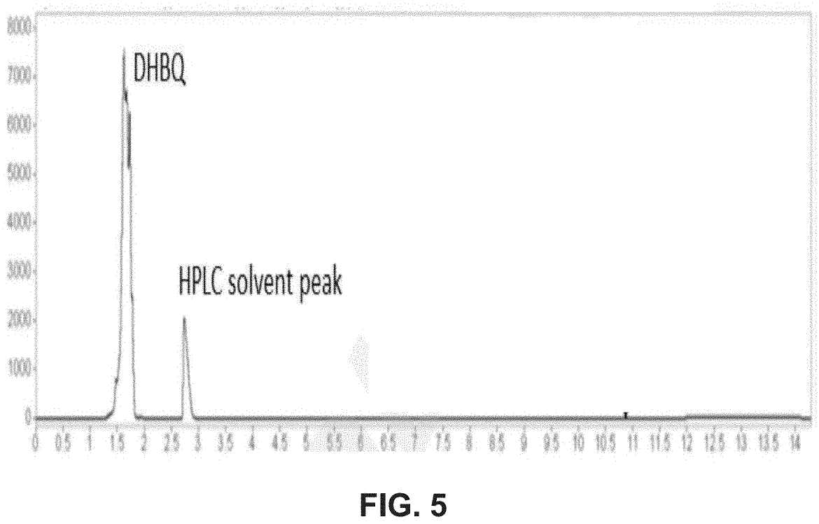

HPLC chromatograms of the input material and the output material from this representative experiment are set out in FIG. 3 and FIG. 4, respectively, and they indicate a substantially clean reaction profile in which THC is converted to CBN. FIG. 4, is also notable in that it does not indicate the presence of DHBQ in the output product. FIG. 5, provides a comparison in this respect, as it sets out an HPLC chromatogram of an unfiltered crude reaction product that indicates a large integration for DHBQ centered at about 1.6 minutes.

Further results from this representative experiment are set out in TABLE 2.

TABLE 2
summary results obtained from the HPLC chromatograms of FIG. 3 and FIG. 4.

| | THC | CBD | CBG | CBC | CBL | CBDV | CBN |
|---|---|---|---|---|---|---|---|
| | (wt. %) | (wt. %) | (wt. %) | (wt. %) | (wt. %) | (wt. %) | (wt. %) |
| Input | 2.61 | 75.57 | 0.70 | 3.23 | 0.41 | 3.05 | 0.29 |
| material | | | | | | | |
| Output | <LoQ | 71.16 | 0.60 | 2.08 | 0.44 | 3.38 | 1.45 |
| material | | | | | | | |

A full factorial experiment design was applied to study the effect of different experimental parameters (i.e. temperature, amount of oxidant, and time) on the oxidation of THC and CBD in the context of the present disclosure. A DOE Matrix for the full factorial experiment design is set out in TABLE 3.

TABLE 3
DOE Matrix for full factorial experiment design for
upgrading low-THC content cannabinoid mixtures.

| | Units | Type | Low | High |
|---|---|---|---|---|
| Temperature | ° C. | Factor | 110 | 140 |
| Equivalent of oxidant | — | Factor | 2 | 10 |
| Time | h | Factor | 7 | 29 |
| dTHC | % | Response | 0 | 100 |
| dCBD | % | Response | 0 | 100 |

The model responses were defined as follows:

$$d T H C = \frac{( THC in output )}{( THC in input )} * 100 and dCBD = \frac{( CBD in output )}{( CBD in input )} * 100$$

Summary results from the full factorial experiment trials are set out in TABLE 4.

TABLE 4
Summary results from the full factorial experiment trials
for upgrading low-THC content cannabinod mixtures.

| | | | Equiv. of | | |
|---|---|---|---|---|---|
| Trials | Time (h) | Temp (° C.) | DHBQ | dTHC (%) | dCBD (%) |
| 1 | 7 | 110 | 2 | 73.2 | 95.4 |
| 2 | 29 | 110 | 2 | 48.6 | 95.0 |
| 3 | 7 | 140 | 2 | 100 | 89.5 |
| 4 | 29 | 140 | 2 | 100 | 92.4 |
| 5 | 7 | 110 | 10 | 58.6 | 94.2 |
| 6 | 29 | 110 | 10 | 28.4 | 81.7 |
| 7 | 7 | 140 | 10 | 100 | 95.0 |
| 8 | 29 | 140 | 10 | 27.2 | 91.7 |

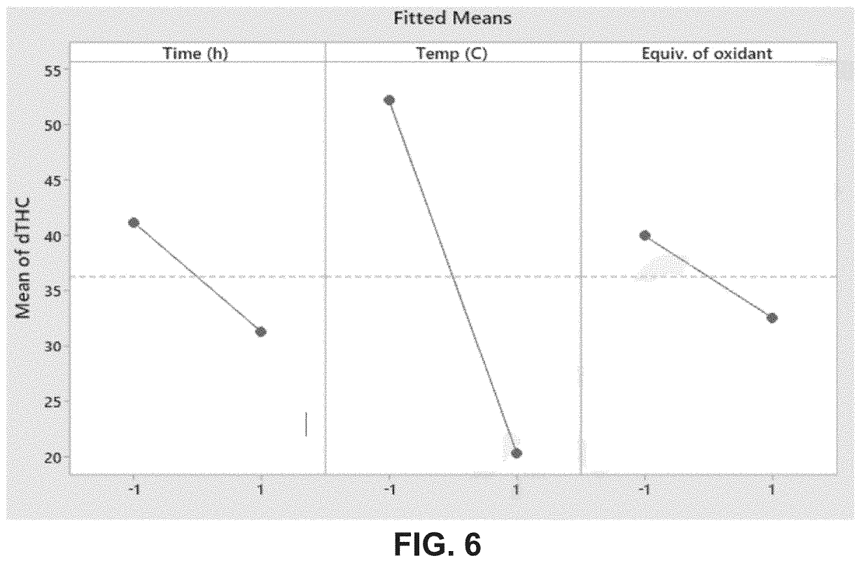

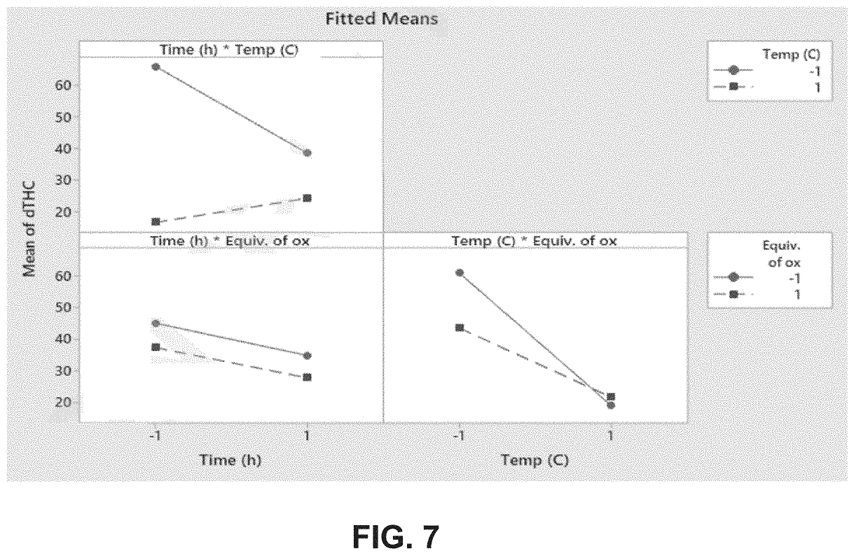

FIG. 6 and FIG. 7 set out the relevant main effect and interaction plots for dTHC, respectively. The results indicate that, under the conditions tested: (i) time, temperature, and DHBQ equivalents all affect dTHC outcomes; (ii) temperature has the strongest effect on dTHC outcomes; and (iii) there is an interaction between temperature and time factors with respect to dTHC outcomes.

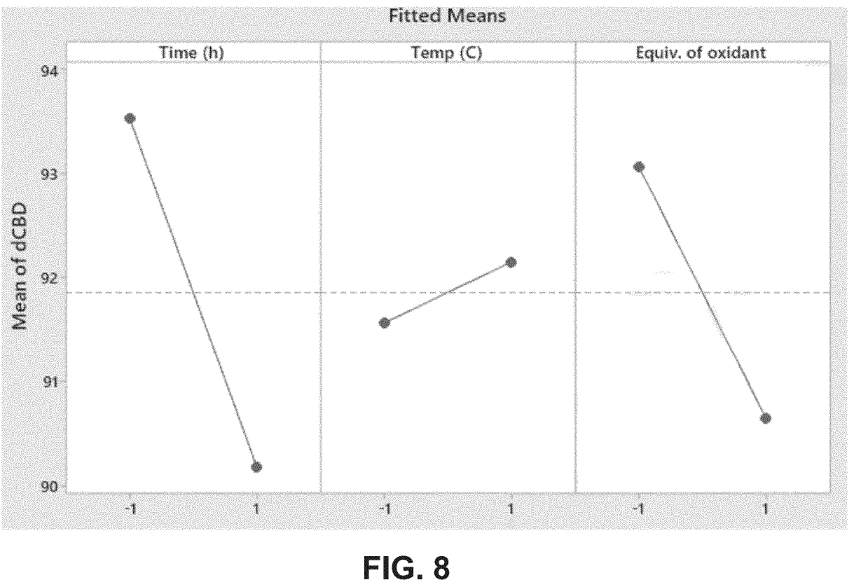

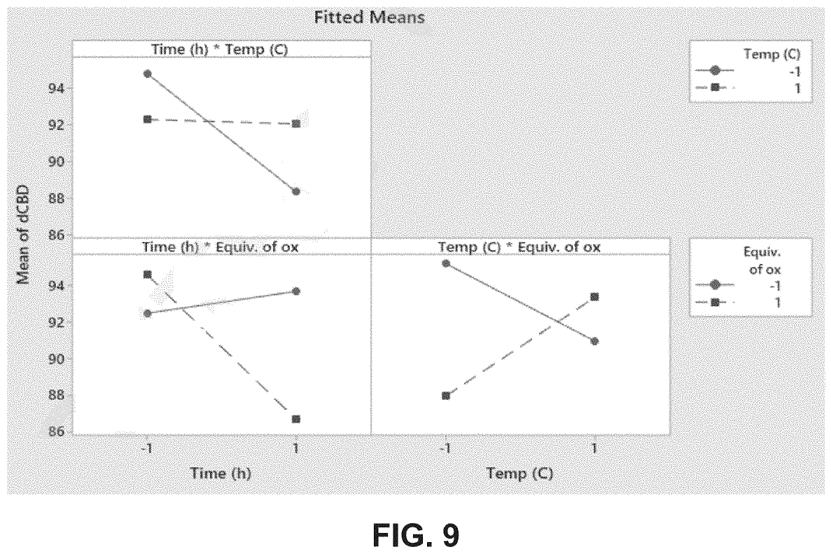

FIG. 8 and FIG. 9 set out the relevant main effect and the interaction plots for dCBD, respectively. The results of FIG. 8 and FIG. 9 indicate that temperature may not be a primary factor in preserving CBD under the conditions evaluated. Instead, the results of FIG. 8 and FIG. 9 indicate that oxidation of CBD depends more heavily on the amount of oxidant and time (as well as interactions between factors).

### Series B

The following examples describe a series of experiments in which complex cannabinoid mixtures having a low THC content were contacted with various benzoquinone reagents to reduce the THC content of the complex cannabinoid mixtures as generally characterized in SCHEME 2.

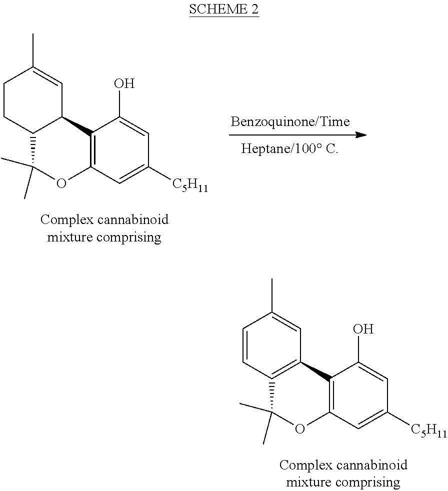

The complex cannabinoid mixture was primarily derived from hemp biomass (referred to as hemp-derived input material below). Analysis by HPLC-DAD indicated that, in advance of the introduction of the benzoquinone reagent, the complex cannabinoid mixture comprised: (i) about 44.4 wt. % CBD; (ii) about 9.5 wt. % THC; (iii) about 0.8 wt. % CBN; (iv) about 5.0 wt. % CBG; and (v) about 11.3 wt. % CBC.

### Example B1

A mixture of the hemp-derived input material, heptane, and tetrachloro-1,4-benzoquinone was stirred and heated to 100° C. for 6 hours to form a crude product mixture. The crude product mixture was cooled to ambient temperature and filtered using a Buchner funnel equipped with a glass frit to separate suspended solids from a filtrate. The filtrate was concentrated in vacuo to provide a crude product residue that was triturated with heptane, filtered using a Buchner funnel equipped with a glass frit, and concentrated in vacuo to provide an upgraded product material. The upgraded product material was analyzed by HPLC-DAD to obtain the results set out in row 2 of TABLE 5.

### Example B2

A mixture of the hemp-derived input material, heptane, and 4-tert-butyl-5-methoxy-1,2-benzoquinone was stirred and heated to 100° C. for 6 hours to form a crude product mixture. The crude product mixture was cooled to ambient temperature and filtered using a Buchner funnel equipped with a glass frit to separate suspended solids from a filtrate. The filtrate was concentrated in vacuo to provide a crude product residue that was triturated with heptane, filtered using a Buchner funnel equipped with a glass frit, and concentrated in vacuo to provide an upgraded product material. The upgraded product material was analyzed by HPLC-DAD to obtain the results set out in row 3 of TABLE 5.

### Example B3

A mixture of the hemp-derived input material, heptane, and thymoquinone was stirred and heated to 100° C. for 18 hours to form a crude product mixture. The crude product mixture was cooled to ambient temperature and filtered using a Buchner funnel equipped with a glass frit to separate suspended solids from a filtrate. The filtrate was concentrated in vacuo to provide a crude product residue that was triturated with heptane, filtered using a Buchner funnel equipped with a glass frit, and concentrated in vacuo to provide an upgraded product material. The upgraded product material was analyzed by HPLC-DAD to obtain the results set out in row 4 of TABLE 5.

TABLE 5
Summary results from EXAMPLES B1, B2, and B3.

| | | | CBN | THC | CBD | CBG | CBC |
|---|---|---|---|---|---|---|---|
| Example | Benzoquinone | Time (h) | yield (%) | recovery (%) | recovery (%) | recovery (%) | recovery (%) |
| 1 | tetrachloro- | 6 | 67 | 34 | 79 | 33 | 61 |
| | 1,4- | | | | | | |
| | benzoquinone | | | | | | |
| 2 | 4-tert-butyl-5- | 18 | 63 | 13 | 75 | 47 | 90 |
| | methoxy-1,2- | | | | | | |
| | benzoquinone | | | | | | |
| 3 | Thymoquinone | 18 | 20 | 23 | 69 | 69 | 71 |

### Series C

The following examples describe a series of experiments in which complex cannabinoid mixtures having low THC contents were contacted with DHBQ to reduce the THC content of the complex cannabinoid mixtures as generally characterized in SCHEME 1.

An exemplary protocol for implementing the transformation of SCHEME 1 in accordance with a method of the present disclosure is as follows.

A reaction vessel was charged with a hemp-derived input material (such as a primary solvent extract or a distillate) and heated to about 80° C. (to reduce its viscosity, for example). DHBQ powder was added to the heated cannabinoid mixture in a quantity sufficient to provide a DHBQ ratio of about 13:1 on a molar basis. The reaction vessel was heated to about 125° C. in the absence of exogenous solvent for about 14 hours with gentle stirring (e.g. 125 rpm). The reaction mixture was filtered hot to obtain crude output material which may be analyzed to determine the quantity of cannabinoids and/or to confirm the presence/absence of DHBQ and/or a reduced form thereof.

### Example C1

Samples of hemp-derived input material were processed according to the above protocol in the absence and presence of atmospheric oxygen. The upgraded product material was analyzed by HPLC-DAD to obtain the results set out in TABLE 6.

TABLE 6
Summary results from EXAMPLE C1.

| Example | Condition | CBD yield (%) | THC yield (%) |
|---|---|---|---|
| 1 | Air only | 101.1 | 91.19 |
| 2 | DHBQ only | 94.2 | 58.62 |
| 3 | DHBQ and air | 94.2 | 8.81 |

### Example C2

Samples of hemp-derived input material were processed according to the above protocol in the presence of atmospheric oxygen. The upgraded product material was analyzed by HPLC-DAD to obtain the results set out in TABLE 7.

TABLE 7
Summary results from EXAMPLE C2.

| | | CBN | CBD | THC | CBG | CBC |
|---|---|---|---|---|---|---|
| Example | Time (h) | yield (%) | recovery (%) | recovery (%) | recovery (%) | recovery (%) |
| 1 | 3 | 36 | 101 | 50 | 74 | 92 |
| 2 | 6 | 40 | 99 | 28 | 73 | 82 |
| 3 | 10 | 47 | 101 | 7 | 97 | 71 |
| 4 | 14 | 78 | 94 | 10 | 110 | 0 |

### Example C3

Samples of hemp-derived input material having varying CBD and THC contents were processed according to the above protocol in the presence of atmospheric oxygen. The upgraded product material was analyzed by HPLC-DAD to obtain the results set out in TABLE 8.

TABLE 8
Summary results from EXAMPLE C3.

| | Input Extract | Input Extract | Output Extract | Output Extract |
|---|---|---|---|---|
| Example | CBD content (%) | THC content (%) | CBD content (%) | THC content (%) |
| 1 | 49.61 | 4.3 | 43.09 | 0.1 |
| 2 | 58.15 | 2.31 | 61.98 | 0.09 |
| 3 | 67.52 | 7.55 | 61.32 | 0.1 |
| 4 | 54.33 | 3.83 | 48.4 | 0.09 |

### Example C4

Samples of hemp-derived input material were processed according to the above protocol at 150° C. in the absence of atmospheric oxygen. The upgraded product material was analyzed by HPLC-DAD to obtain the results set out in TABLE 9.

TABLE 9
Summary results from EXAMPLE C4.

| | | Equiv. | CBN | CBD | THC | CBG | CBC |
|---|---|---|---|---|---|---|---|
| Example | Time (h) | of DHBQ | yield (%) | recovery (%) | recovery (%) | recovery (%) | recovery (%) |
| 1 | 7 | 2 | 574 | 50.5 | 25.2 | 0.0 | 0.0 |
| 2 | 7 | 10 | — | 97.2 | 60.9 | — | — |
| 3 | 18 | 1 | 70.3 | 92.8 | 33.7 | 85.7 | 78.9 |

### Example C5

Samples of hemp-derived input material were processed according to the above protocol in the presence of atmospheric oxygen and crystallized to produce crystalline CBD. Crystalline CBD obtained from the hemp-derived input material and the crystalline CBD obtained from the upgraded product material was analyzed by HPLC-DAD to obtain the results set out in TABLE 10.

TABLE 10
Summary results from EXAMPLE C5.

| | CBD | CBD | CBDV | THC |
|---|---|---|---|---|
| Crystals | crystallization | content | content | content |
| obtained from: | yield (%) | (%) | (%) | (ppm) |
| Input-material | 52 | >97 | 2.41 | 202.2 |
| Upgraded | 55 | >97 | 2.20 | 26.5 |
| product | | | | |

### Series D

The following examples describe a series of experiments in which a cannabinoid distillate having low THC contents was contacted with DHBQ to reduce the THC content of the cannabinoid distillate as generally characterized in SCHEME 1.

An exemplary protocol for implementing the transformation of SCHEME 1 in accordance with a method of the present disclosure is as follows.

A reaction vessel was charged with a hemp-derived distillate. DHBQ powder was added to the cannabinoid distillate in a quantity sufficient to provide a DHBQ ratio of about 13:1 or about 6.7:1 on a molar basis. The reaction vessel was heated to about 112° C. in the absence of exogenous solvent in a Parr reactor with agitation for up to 24 hours. The reaction mixture was filtered hot to obtain crude output material which may be analyzed to determine the quantity of cannabinoids and/or to confirm the presence/absence of DHBQ and/or a reduced form thereof.

### Example D1

Samples of hemp-derived distillate were processed according to the above protocol in the presence of atmospheric oxygen. The upgraded product material was analyzed by HPLC-DAD to obtain the results set out in TABLE 11.

TABLE 11
Summary results from EXAMPLE D1.

| | | DHBQ:THC molar ratio | | DHBQ:THC molar ratio about 6.7:1; | | | |
|---|---|---|---|---|---|---|---|
| Distillate | | about 13:1; with air | | with air | | | |
| composition | | 13 h | 24 h | 3 h | 6 h | 13 h | 24 h |
| CBD | 45.5 | 39.2 | 38.1 | 41.52 | 43.89 | 39.6 | 33 |
| THC | 1.51 | 0.08 | 0.23 | 0.9 | 0.12 | 0.07 | 0.18 |
| CBC | 2.56 | 1.47 | 0.32 | 2.25 | 1.96 | 0.99 | 0.44 |

### Example D2

Samples of hemp-derived distillate were processed according to the above protocol at 125° C. in the presence of atmospheric oxygen with DHBQ at a DHBQ:THC molar ratio of about 6.7:1.0. The upgraded product material was analyzed by HPLC-DAD to obtain the results set out in TABLE 12.

TABLE 12
Summary results from EXAMPLE D2.

| | | DHBQ:THC molar ratio about 6.7:1; with air | | |
|---|---|---|---|---|
| Distillate composition | | 3 h | 6 h | 23 h |
| CBDVA | 0.17 | 0.2 | 0.26 | 0.39 |
| CBDV | 2.02 | 1.09 | 1.09 | 1.01 |
| CBG | 0.45 | 0.28 | 0.25 | 0.22 |
| CBD | 45.25 | 42.88 | 40.97 | 38.59 |
| CBN | 0.15 | 0.56 | 0.82 | 1.17 |
| Δ9-THC | 1.80 | 0.09 | 0.17 | 0.21 |
| Δ8-THC | <0.01 | 0.03 | 0.05 | 0.1 |
| CBC | 2.99 | 2.03 | 1.73 | 1.06 |
| Δ9-THCA | <0.01 | <0.01 | <0.01 | <0.01 |
| TOTAL | 52.89 | 47.62 | 45.80 | 43.21 |

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/-10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of” or “consist of” the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are discussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1. A method for reducing the tetrahydrocannabinol (THC) content in a composition comprising THC, the method comprising:
contacting the composition comprising THC with a benzoquinone reagent, optionally in the presence of a solvent.

2.-6. (canceled)

7. The method of claim 1, wherein the composition comprising THC is derived from a hemp biomass.

8. The method of claim 1, wherein the composition comprising THC is a cannabis distillate, a cannabis resin, a cannabis extract, or a combination thereof.

9. The method of claim 1, wherein the benzoquinone reagent comprises a compound as defined in formula (I) or formula (II): 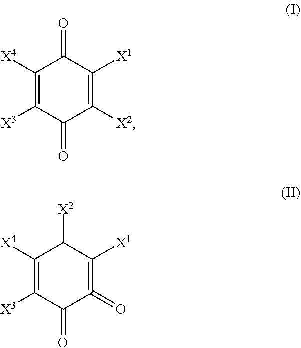
wherein X¹, X², X³, and X⁴ are each independently: H; a halide; a C_{<12}-hydrocarbyl; a C_{<12}-heteroaryl; a C_{<12}-heteroaralkyl; a C_{<12}-heteroaralkenyl; hydroxyl; a C_{<12}-alkoxy; a C_{<12}-amino; a C_{<12}-acyl; a C_{<12}-amide; a C_{<12}-ester; a C_{<12}-ketone; or a substituted analog thereof.

10. The method of claim 1, wherein the benzpquinone reagent comprises: 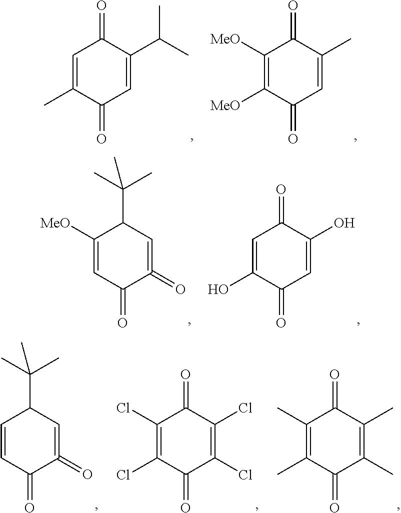
or a combination thereof.

11. (canceled)

12. (canceled)

13. The method of claim 1, wherein the contacting of the composition comprising THC with the benzoquinone reagent is at a benzoquinone:THC ratio of between about 1.0:1.0 and about 13.0:1.0 on a molar basis.

14. The method of claim 1, wherein the contacting of the composition comprising THC with the benzoquinone reagent is at a benzoquinone:THC ratio of between about 2.5:1.0 and about 7.0:1.0 on a molar basis.

15. The method of claim 1, wherein the contacting of the composition comprising THC with the benzoquinone reagent is performed at a temperature of between about 20° C. and about 180° C.

16. The method of claim 1, wherein the contacting of the composition comprising THC with the benzoquinone reagent is performed at a temperature of between about 100° C. and about 130° C.

17. (canceled)

18. (canceled)

19. The method of claim 1, wherein the contacting of the composition comprising THC with the benzoquinone reagent is in the presence of the solvent.

20. The method of claim 19, wherein the solvent is pentane, hexane, heptane, methanol, ethanol, isopropanol, dimethyl sulfoxide, acetone, ethyl acetate, diethyl ether, tert-butyl methyl ether, water, acetic acid, anisole, 1-butanol, 2-butanol, butane, butyl acetate, ethyl formate, formic acid, isobutyl acetate, isopropyl acetate, methyl acetate, 3-methyl-1-butanol, methylethyl ketone, 2-methyl-1-propanol, 1-pentanol, 1-propanol, propane, propyl acetate, trimethylamine, or a combination thereof.

21. (canceled)

22. (canceled)

23. The method of claim 1, wherein the THC content of the composition comprising THC is reduced to less than 0.3% by performing the method.

24. A method for reducing the tetrahydrocannabinol (THC) content in a composition comprising THC and cannabidiol (CBD), the method comprising:
contacting the composition comprising THC and CBD with 2,5-dihydroxy-1,4-benzoquinone, 4-tert-butyl-5-methoxy-1,2-benzoquinone, tetrachloro-1,4-benzoquinone or thymoquinone; such that the THC content reduced to a greater extent than the CBD content on a relative wt. % reduction basis.

25.-27. (canceled)

28. The method of claim 1, wherein prior to performing the method, the composition comprising THC has a THC content of less than about 20 wt. %.

29. The method of claim 9, wherein the benzoquinone reagent comprises:
a compound as defined in formula (I) where X¹═H, X²═H, X³═H, and X⁴═H,
a compound as defined in formula (I) where X¹═CN, X²═CN, X³═Cl, and X⁴═Cl,
a compound as defined in formula (II) where X¹═H, X²═C(CH₃)₃, X³═C(CH₃)₃, and X⁴═H,
a compound as defined in formula (II) where X¹═Cl, X²═Cl, X³═Cl, and X⁴═Cl,
a compound as defined in formula (I) where X¹═Cl, X²═Cl, X³═Cl, and X⁴═Cl,
a compound as defined in formula (II) where X¹═H, X²═C(CH₃)₃, X³═H, and X⁴═H,
a compound as defined in formula (I) where X¹═H, X²═OH, X³═H, and X⁴═H,
a compound as defined in formula (II) where X¹═H, X²═C(CH₃)₃, X³═H, and X⁴═OCH₃, or
a compound as defined in formula (II) where X¹═H, X²═H, X³═H, and X⁴═OCH₃.

30. The method of claim 19, wherein the solvent is a protic solvent.

31. The method of claim 19, wherein the solvent is an aprotic solvent.

32. The method of claim 24, further comprising contacting the composition comprising THC and CBD with 2,5-dihydroxy-1,4-benzoquinone or thymoquinone at a temperature of between about 80° C. and about 190° C.

33. The method of claim 24, further comprising contacting the composition comprising THC and CBD with 4-tert-butyl-5-methoxy-1,2-benzoquinone at a temperature of between about 70° C. and about 160° C.

34. The method of claim 24, which comprises contacting the composition comprising THC and CBD with tetrachloro-1,4-benzoquinone at a temperature of between about 80° C. and about 180° C.
